(19) United States
(12) Patent Application Publication
(10) Pub. No.: US 2021/0290439 A1
(43) Pub. Date: Sep. 23, 2021

(54) Goggle Loupes

(71) Applicant: Wei Liu, Holyoke, MA (US)

(72) Inventor: Wei Liu, Holyoke, MA (US)

(21) Appl. No.: 17/163,816

(22) Filed: Feb. 1, 2021

Related U.S. Application Data
(60) Provisional application No. 62/992,207, filed on Mar. 20, 2020.

(51) Int. Cl. A61F 9/02; G02B 25/00; G02C 11/04

(52) U.S. Cl. CPC A61F 9/029; G02C 11/04; G02B 25/004

(57) ABSTRACT

A device includes the elements of full eye coverage goggles and magnifying binocular loupes. So wearers have eye protection against small particles, such as virus when doing delicate procedures requiring magnification to fine details. The device could work with other personal protective equipment to reach higher level protection.

### CROSS-REFERENCE TO RELATED APPLICATIONS

Related provisional application No. 62/992,207; confirm No. 2805; EFS ID 38921919

### STATEMENT REGARDING FEDERALLY SPONSORED RESEARCH OR DEVELOPMENT (IF APPLICABLE)

Not Applicable

### REFERENCE TO SEQUENCE LISTING (IF APPLICABLE), A TABLE, OR A COMPUTER PROGRAM LISTING COMPACT OISE APPENDIX (IF APPLICABLE)

Not Applicable

### BACKGROUND OF THE INVENTION

The challenges to eyewear are that wearers (including but not limited to surgeons, dentists and other care providers) need magnification to see fine details while performing delicate procedures with full coverage eye protection against small particles, such as airborne microorganisms introducing into human body through the mucous membranes of eye (conjunctiva) via contamination in the air. High level isolation suit and microscope for every procedure are expensive and lack of dexterity. In addition, current common personal protective equipment is not likely to reach higher level protection effectively and efficiently.

### BRIEF SUMMARY OF THE INVENTION

By providing a device having full eye coverage and loupes, protection to wearers and magnifying function are ensured at the same time. Further, by providing this device being able to be combined with other personal protective equipment (respirator, isolation suit or gown, etc.), higher levels of protection could be reached with improved flexibility and cost efficiency.

The disclosed exemplary embodiment provides a device which has at least two major portions—a loupe member and a goggle member, including a seal member and a stabilization member. This eyewear also comprises a headlight member.

Following detailed description will make the present application apparent regarding further objects, features, advantages and properties of device.

### BRIEF DESCRIPTION OF THE DRAWING

In the following portion of the present description, the teachings of the present applications would be explained in more detail with reference to the example embodiment shown in the drawing, in which:

FIG. 1 is an isometric view of a device according to an embodiment

### DETAILED DESCRIPTION OF THE INVENTION

In the following detailed description, the device according to the teachings for this application in the form of eyewear will be described by the embodiment.

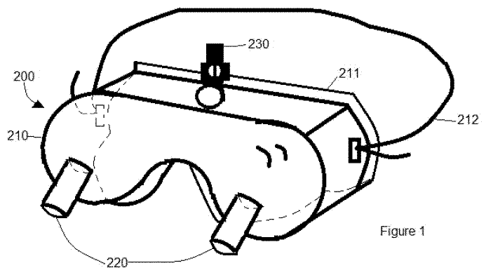

An embodiment of the eyewear 200 is illustrated in FIG. 1. The eyewear 200 comprises a goggle member 210, a loupe member 220 and a headlight member 230. The goggle member 210 comprises a seal member 211 along the edge of goggle contacting skin around wearer's eye area and band member 212 for stabilizing the eyewear on wearer's head.

The goggle member 210 connects other members for in-placed functions and extended functions if needed. Such as a holder for mounting a headlight 230 is presented and a holder for a mini camera could be added.

The loupe member 220 is installed directly on goggle member 210 as binocular loupes. The loupe member is custom-made for wearers.

In this embodiment the seal member 211 can be pre-made or custom-made according to wearer to reach different levels of isolation to eye area and/or meet other needs.

Also, the seal member 211 could be fixed or removable to goggle member 210. Such as a seal member based on silicon related material is custom-made according to one wearer to make the goggle member air-tight for this wearer.

The band member 212 is for stabilization of the eyewear 200 and could be replaced by other device for stabilization and other purposes.

In the present embodiment features of the eyewear 200 are dynamic and driven by purposes and functions. For examples, the rear part of goggle member 210 could flare out more for connecting with other personal protective equipment to reach more overall protection; the rear edge of goggle member 210 could have a curvature following wearer's face to reach tighter sealing result; transparent material is used for goggle member 210 for wide range of vision; anti-fog material and/or device is adopted for keeping clear view; prescription glasses are installed directly or indirectly via device on eyewear 200.

The various aspects of what is described above could be used alone or in different combinations. It should be noted that this application is not limited to the use itself but can be used with other protective equipment. In addition, a seal or device could be used to remove gaps among different protective equipment parts to improve protection level, such as a gap (showing skin of a wearer) between goggle loupes and a mask.

The teaching of the present application has many advantages. Various embodiments or implementations may yield one or more of the following advantages. It should be noted that this is not an exhaustive list and there may be other advantages that are not described herein. For example, one advantage of the teaching of this application is it provides wearers full coverage eye protection and magnification to fine details at the same time. It is another advantage of the present invention that it provides custom-made seal to wears' eye area. It is yet another advantage of the present invention that it provides for a device with ability to work with other protective equipment to improve overall protection effectively and efficiently.

Although the teaching of the present application has been described in detail for purpose of illustration, it is understood that such detail is solely for that purpose, and variations can be applied therein skilled in the art without departing from the scope of the teaching of this application. For example, the device has been described with a wireless headlight, but it is understood that the headlight does not need to be wireless, and it could be headlight with wire and portable battery.

Features described in the preceding description may be used in combination other than the combinations explicitly described.

Whilst endeavoring in the foregoing specification to draw attention to those features of the invention believed to be of particular importance it should be understood that the applicant claims protection in respect of any patentable feature or combination of features hereinbefore referred to and/or shown in the drawing whether or not particular emphasis has been placed thereon.

The term “comprising” as used in the claim session does not exclude other elements or steps. The term “a” or “an” as used in the claim session does not exclude a plurality. A unit or other means may fulfill the functions of several units or means recited in the claim session.

1. An eyewear device comprising of:
a goggle member having a front portion, a top portion and a rear portion;
a binocular loupe member secured to said front portion of said goggle member.

2. An eyewear device as in claim 1 is further comprising of a headlight member removably secured on said top portion of said goggle member

3. An eyewear device as in claim 1, wherein said rear portion of said goggle member can install a seal member.

4. A seal member as in claim 3 could be custom-made.

5. An eyewear device as in claim 1, wherein said rear portion has a flange.

6. An eyewear device as in claim 1, could work with other personal protective equipment such as mask, isolation suit or gown to reach different level of protection directly or indirectly.
